(19) United States
(12) Patent Application Publication
(10) Pub. No.: US 2006/0233833 A1
(43) Pub. Date: Oct. 19, 2006

(54) Novel uses of parapoxvirus preparations

(75) Inventors: Hans-Robert Hehnen, Siegburg (DE); Bernhard Kaltenboeck, Auburn, AL (US); Tobias Schlapp, Isernhagen (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, LEVERKUSEN (DE)

(21) Appl. No.: 10/515,532

(22) PCT Filed: May 23, 2003
(86) PCT No.: PCT/EP03/05397

§ 371 Date: Oct. 3, 2005

Related U.S. Application Data
(60) Provisional application No. 60/383,988, filed on May 29, 2002.

(51) Int. Cl. A61K 39/275; A61K 39/285

(52) U.S. Cl. 424/232.1

(57) ABSTRACT

The present invention related to use of Parapoxvirus preparations for the treatment of conditions related to infections with strictly intracellular bacteria

### FIELD OF THE INVENTION

The present invention relates to use of Parapoxvirus preparations for the treatment of conditions related to infections with Chlamydia and other strictly intracellular bacteria.

### BACKGROUND OF THE INVENTION

It is known from several in vivo studies that the prophylactic application of Parapoxvirus preparations can strengthen the immune response. This therapeutic efficacy has been applied to infections with virus as well as bacteria in animal health. For example, the use of Parapoxvirus preparations in bovine mastitis due to Staphylococcus aureus infection showed a significant reduction of infection (Zecconi et al., 1999). Likewise, immunostimulation of leukocytes have been described in animals as well as humans (Yirrell et al., 1994; Haig et al., 1996; Haig et al., 1999). The main mechanism of the stimulation detected is the induction of different cytokines (e.g., interferons α or γ, diverse interleukins). In summary, the published results showed that Parapoxvirus preparations stimulates cells of the immune system by increasing the amount of soluble mediators.

BAYPAMUN®, a pharmaceutical product, is used to induce “paraspecific immunity,” i.e., for inducing the unspecific immune system. It is used therapeutically, metaphylactically, and prophylactically for the treatment of animals in need. BAYPAMUN® is manufactured from chemically inactivated Parapoxvirus ovis strain D1701 (German Patent DE3504940). The inactivated Parapoxvirus ovis induces in animals non-specific protection against infections with a wide variety of extracellular pathogens. It is assumed that this protection is mediated via various mechanisms in the body's own defense system. These mechanisms include the induction of interferons, the activation of natural killer cells, the induction of “colony-stimulating activity” (CSA), apoptosis, and the stimulation of lymphocyte proliferation. Earlier investigations of the mechanism of action demonstrated the stimulation of interleukin-2 and interferon-α.

New Zealand patent application No. 512341 (filed Jul. 13, 2001) discloses that individual viral proteins of Parapoxvirus ovis stain NZ2 and groups of NZ2 proteins can mimic the effect of preparation of the full virus particle.

Also known is that Parapoxvirus preparations can be used to treat infections with extracellular bacteria. What is not previously known is that Parapoxvirus preparations can be used to treat infections with strictly intracellular bacteria, such as Chlamydia. This finding is surprising and unexpected since the human or animal body's defense mechanisms against strictly intracellular bacteria is very different from the body's defense mechanism against extracellular pathogens and bacteria.

The human and animal immune system protects the human or animal body from invasion of microbial cells. The so-called first-line-of-defense are cells of the innate immune system, mainly granulocytes and macrophages. Both cell types attack and phagocytose microbial pathogens directly. However, they need further support from the specific immune system. T-cells and B-cells are the most prominent members of the so-called specific immune response. B-cells produce specific antibodies which specifically detect, opsonize and neutralize microbial invaders. T-cells are the major players for the production and release of specific soluble factors, which activate other immune effector cells. Another specific function of T-cells is the killing of infected or otherwise abnormal cells in the body (e.g., tumors). The mechanism of antibody opsonization and direct attack cannot work for strictly intracellular bacteria as they are not floating freely in the blood. Due to their intracellular nature, these pathogens can be indirectly attacked by the immune system. In this context, the T-cells mediate an effective immune response (e.g., via the secretion of specific mediators like interferons). This process lead to an activation of the cells of the innate immune system, enabling them to overcome the intracellular pathogens by killing them intracellularly. On the other hand, the T-cells recognize the infected cells and mediate lysis or cell death. Due to release of the pathogens from their intracellular habitat, they are thereafter accessible to different defense mechanisms.

Intracellular bacteria can be divided into 2 main groups: firstly, the facultative intracellular bacteria (like Listeria, Mycobacteria, Salmonella, and Legionella) able to grow also outside the eukaryotic host cell, and secondly, strictly intracellular species (such as, e.g., Chlamydia spec., Chlamydia pneumoniae, Chlamydia psittaci, Chlamydia trachomatis) strictly dependent on intracellular growth. All intracellular pathogens are also known to infect domestic animals, thereby mediating different pathologies. Intracellular bacteria are in general difficult in therapy.

Due to the nature of strictly intracellular bacteria and their respective habitat (e.g., phagosome, cytoplasm), antibiotics are very limited in the ability to achieve resolution of an infection. The antibiotic used in therapy must enter the host cell and remain active. Due to the chemical nature of some antibiotics, this is achieved and these antibiotics (e.g., quinolones, macrolides) are used in infections with intracellular pathogens, although with limited efficacy. The pathology of intracellular bacteria is mainly a chronic one, leading to even more difficult therapeutic efficacy.

It is known to a person skilled in the art that conditions, such as pneumonia (Grayston et al., 1989), arteriosclerosis (Leinonen, 2000), multiple sclerosis (Moses and Sriram, 2001), arthritis (Inman et al., 2000), and asthma (Cook, 1999) can all be related to infections with strictly intracellular bacteria.

Hence, it is desirable to have available methods or pharmaceutical compositions to prophylactically or therapeutically treat infections with strictly intracellular bacteria and diseases and conditions related to these infections in humans as well as in non-human animals.

### SUMMARY OF THE INVENTION

The present invention relates to the use of Parapoxvirus preparations for the prophylaxis or therapy of infections with strictly intracellular bacteria. In a preferred embodiment, the invention relates to the use of Parapoxviruses preparations (e.g., preparations of Parapoxvirus ovis, Parapoxvirus ovis strain D1701, Parapoxvirus ovis strain NZ2, Parapoxvirus ovis strain NZ7, Parapoxvirus ovis strain NZ10, orf virus, or orf-11 virus) for the prophylaxis or treatment of infections with strictly intracellular bacteria. The present invention also relates to pharmaceutical compositions and methods of manufacture of pharmaceutical compositions, comprising Parapoxvirus preparations, useful in the treatment of infections with strictly intracellular bacteria.

In a preferred embodiment of the invention, the strictly intracellular bacterial infection to be treated is an infection with Chlamydia. In a more preferred embodiment of the invention, the strictly intracellular bacterial infection to be treated is an infection with Chlamydia pneumoniae, Chlamydia psittaci, and/or Chlamydia trachomatis. Infections with Chlamydia are known to lead to other diseases and pathological states, such as, e.g., pneumonia, arteriosclerosis, arteriosclerosis, arthritis, asthma, and other diseases. It is well known to persons skilled in the art that conditions, such as pneumonia (Grayston et al., 1989), arteriosclerosis (Leinonen, 2000), multiple sclerosis (Moses and Sriram, 2001), arthritis (Inman et al., 2000), and asthma (Cook, 1999) can all be related to infections with strictly intracellular bacteria. One embodiment of the invention relates to the treatment of these diseases and pharmaceutical compositions for use in the treatment or prophylaxis of these diseases.

### BRIEF DESCRIPTION OF THE DRAWINGS

FIG. 1 shows lung weights of mice 12 days after intranasal infection with 2×10⁶ IFU Chlamydia psittaci B577 and treatment with BAYPAMUN® in the early phase of the infection. Error bars indicate the standard error of the mean. The lung weight increase of infected mice over that of naive, unchallenged mice is a direct measure of disease intensity at this time after inoculation.

### DETAILED DESCRIPTION OF THE INVENTION

“Conditions related to infection with strictly intracellular bacteria,” within the meaning of the invention, are diseases and conditions which are related to or caused by infections with strictly intracellular bacteria. Such conditions, within the meaning of the invention are, e.g., but not limited to, pneumonia, arteriosclerosis, multiple sclerosis, arthritis, and asthma. Infection with strictly intracellular bacteria itself is also regarded to be “a condition related to infection with strictly intracellular bacteria.”

A “protein,” within the meaning of the invention, is any polypeptide of at least five amino acids.

“Recombinant Parapoxvirus protein,” within the meaning of the invention, is any protein encoded by a Parapoxvirus genome, that is expressed by or in a cell, to which cell the coding polynucleotide was introduced using recombinant DNA technology. Recombinant DNA technology encompasses the use of bacterial vectors, viral vectors, other vectors, DNA molecules, and other agents for transferring nucleic acids into a host cell. Other techniques of recombinant DNA technology relating to the invention, such as electroporation, the use of competent cells, and other techniques are well known to a person skilled in the art.

“Parapoxvirus preparations,” within the meaning of the invention, is understood as being any biological material which is obtained from, or present in, any member of the Parapoxvirus genus. In a preferred embodiment of the invention, Parapoxvirus preparations also comprise recombinant proteins encoded by a Parapoxvirus genome. These recombinant proteins can be used alone or in any combination.

Parapoxvirus preparations can contain biological material obtained from or coded by the genome of more than one member of the Parapoxvirus genus. In a preferred embodiment of the invention, Parapoxvirus preparations also comprise suitable carriers and/or adjuvants and other substances that are useful in the preparation of a pharmaceutical composition. In a preferred embodiment of the invention, the Parapoxvirus preparation contains only inactivated biological material.

New Zealand patent application No. 512341 (filed Jul. 13, 2001) discloses that individual viral proteins of Parapoxvirus ovis stain NZ2 and groups of NZ2 proteins can mimic the effect of preparation of the full virus particle. Likewise, Parapoxvirus proteins or recombinant Parapoxvirus proteins are effective individually and in combination with other Parapoxvirus proteins or recombinant Parapoxvirus proteins for the treatment of conditions related to infection with strictly intracellular bacteria.

German Patent DE3504940 (published on Nov. 9, 1997) contains a detailed description of methods for the manufacture of Parapoxvirus preparations. Other methods for the manufacture of Parapoxvirus preparations are known to a person skilled in the art.

The present invention relates to the use of Parapoxvirus preparations for the prophylaxis or therapy of infections with strictly intracellular bacteria and conditions related to such infections. In a preferred embodiment of the invention, the invention relates to the use of Parapoxvirus preparations comprising Parapoxvirus material from Parapoxviruses (such as, e.g., Parapoxvirus ovis, Parapoxvirus ovis strain D1701, Parapoxvirus ovis strain NZ2, Parapoxvirus ovis strain NZ7, Parapoxvirus ovis strain NZ10, orf virus, orf-11 virus) for the prophylaxis or treatment of infections with strictly intracellular bacteria.

In a preferred embodiment of the invention, the Parapoxvirus preparation contains only inactivated biological material. Parapoxvirus material can be inactivated by methods well known to a person skilled in the art, such as, e.g., by radiation.

In a preferred embodiment of the invention, the strictly intracellular bacterial infection to be treated is an infection with Chlamydia. Infections with Chlamydia are related to other diseases and pathological states, such as, e.g., but not limited to, pneumonia, arteriosclerosis, multiple sclerosis, arthritis, and asthma. One embodiment of the invention relates to the treatment of these diseases and to pharmaceutical compositions for use in the treatment or prophylaxis of these diseases.

Parapoxvirus and recombinant Parapoxvirus proteins can be administered systemically (e.g., intravenously (i.v.), subcutaneously, intramuscularly, intracutaneously, intraperitoneally), locally, or orally (per os). The recombinant proteins or products thereof should be formulated appropriately, e.g. in a non-pyrogenic solution or suspension for i.v. use, or in capsules for implantation, or in capsules for per os use. Pharmaceutical compositions of the invention can be administered, e.g., oral, nasal, anal, vaginal etc., as well as parenteral administration. Pharmaceutical compositions of the invention can be in the form of suspensions, solutions, syrups, elixirs, or appropriate formulations in polymers as well as liposomes.

The invention also relates to recombinant Parapoxvirus proteins. Recombinant Parapoxvirus proteins of the invention can be prepared, e.g., with suitable recombinant cell lines. Alternatively, non-recombinant cell lines, such as WI-38, MRC-5, or Vero cells, can be infected with recombinant viruses that carry the recombinant genes using viral vectors such as, but not limited to, the Vaccina virus (e.g., Vaccina lister). In addition, other suitable viruses can be used in combination with other suitable cells (e.g., using Vaccinia virus vectors and fibroblasts as host cells or baculovirus vectors and insect cells as host cells). It is advantageous to cultivate the recombinant cell cultures in high-cell-density fermentations to achieve favorable productivity and a good overall process performance.

The present invention relates to the use of Parapoxvirus preparations for the manufacture of a pharmaceutical composition for the prophylaxis or for the treatment of conditions related to infection with strictly intracellular bacteria.

In a preferred embodiment, the invention further relates to the use of Parapoxvirus preparations for the manufacture of a pharmaceutical composition for the prophylaxis or for the treatment of conditions related to infection with strictly intracellular bacteria, wherein the infection is Chlamydia.

In a preferred embodiment, the invention further relates to the use of Parapoxvirus preparations for the manufacture of a pharmaceutical composition for the prophylaxis or for the treatment of conditions related to infection with strictly intracellular bacteria, wherein the Parapoxvirus preparation comprises material from a Parapoxvirus ovis, or from a Parapoxvirus ovis strain NZ2, or from a Parapoxvirus ovis strain NZ7, or from a Parapoxvirus ovis strain NZ10, or from a Parapoxvirus ovis strain D1701, or from an orf virus, or an orf-11 virus.

In a preferred embodiment, the invention further relates to the use of Parapoxvirus preparations for the manufacture of a pharmaceutical composition for the prophylaxis or for the treatment of conditions related to infection with strictly intracellular bacteria, wherein the condition related to infection with strictly intracellular bacteria is arteriosclerosis, and/or pneumonia, and/or multiple sclerosis, and/or asthma, and/or arthritis.

In a preferred embodiment, the invention further relates to the use of Parapoxvirus preparations for the manufacture of a pharmaceutical composition for the prophylaxis or for the treatment of conditions related to infection with strictly intracellular bacteria, wherein the Parapoxvirus preparation comprises recombinant Parapoxvirus protein.

The invention further relates to a method of treatment of conditions related to infections with strictly intracellular bacteria by administering to a subject in need a therapeutically effective dose of a Parapoxvirus preparation.

In a preferred embodiment, the invention further relates to a method of treatment of conditions related to infections with strictly intracellular bacteria by administering to a subject in need a therapeutically effective dose of a Parapoxvirus preparation, wherein the infection is Chlamydia.

In a preferred embodiment, the invention further relates to a method of treatment of conditions related to infections with strictly intracellular bacteria by administering to a subject in need a therapeutically effective dose of a Parapoxvirus preparation, wherein the Parapoxvirus preparation comprises material from Parapoxvirus ovis, or from a Parapoxvirus ovis strain NZ2, or from a Parapoxvirus ovis strain NZ7, or from a Parapoxvirus ovis strain NZ10, or from a Parapoxvirus ovis strain D1701, or from an orf virus, or an orf-11 virus.

In a preferred embodiment, the invention further relates to a method of treatment of conditions related to infections with strictly intracellular bacteria by administering to a subject in need a therapeutically effective dose of a Parapoxvirus preparation, wherein the condition related to infection with strictly intracellular bacteria is arteriosclerosis, and/or pneumonia, and/or multiple sclerosis, and/or asthma, and/or arthritis.

In a preferred embodiment, the invention further relates to a method of treatment of conditions related to infections with strictly intracellular bacteria by administering to a subject in need a therapeutically effective dose of a Parapoxvirus preparation, wherein the Parapoxvirus preparation comprises recombinant Parapoxvirus protein.

The invention further relates to a pharmaceutical composition for use in the treatment of prophylaxis of conditions related to infections with strictly intracellular bacteria, wherein said pharmaceutical composition comprises a Parapoxvirus preparation.

In a preferred embodiment, the invention further relates to a pharmaceutical composition for use in the treatment of prophylaxis of conditions related to infections with strictly intracellular bacteria, wherein said pharmaceutical composition comprises a Parapoxvirus preparation, wherein the infection is Chlamydia.

In a preferred embodiment, the invention further relates to a pharmaceutical composition for use in the treatment of prophylaxis of conditions related to infections with strictly intracellular bacteria, wherein the Parapoxvirus preparation comprises material from a Parapoxvirus ovis, or a Parapoxvirus ovis strain NZ2, or a Parapoxvirus ovis strain NZ7, or a Parapoxvirus ovis strain NZ10, or a Parapoxvirus ovis strain D1701, or an orf virus, or an orf-11 virus.

In a preferred embodiment, the invention further relates to a pharmaceutical composition for use in the treatment of prophylaxis of conditions related to infections with strictly intracellular bacteria, wherein the condition related to infection with strictly intracellular bacteria is arteriosclerosis, and/or pneumonia, and/or multiple sclerosis, and/or asthma, and/or arthritis.

In a preferred embodiment, the invention further relates to a pharmaceutical composition for use in the treatment of prophylaxis of conditions related to infections with strictly intracellular bacteria, wherein the Parapoxvirus preparation comprises recombinant Parapoxvirus protein.

### EXAMPLES

### Example 1

The effect of Parapoxvirus on the response of mice to challenges with Chlamydia psittaci (C. psittaci) B577 was investigated.

Mice of the group “live C. psittaci B577 vaccinated, challenged” served as controls for optimum protection and received a low-level intranasal infection with 3×10⁴ inclusion forming units (IFU) of C. psittaci in 20 microliters sucrose-phosphate-glutamate (SPG) buffer. This infection typically confers complete resistance to subsequent homologous challenge in BALB/c mice.

Four weeks later, all groups except “naive, non-challenged” were challenged intranasally with a high dose of 3×10⁶ IFU C. psittaci B577. This is approx. equivalent to an LD₂₀ 12 days after inoculation. LD₂₀ is the dose that leads to the death of approx. 20% of the test animals.

BAYPAMUN® treated groups received 3 intraperitoneal injections: 16 hours prior to challenge, 48 hours later, and 96 hours later, each 100 microliters of BAYPAMUN® dissolved in H₂O, or further diluted in PBS 1:6 or 1:36 (“Baypamun undiluted, challenged,” “Baypamun diluted 1:6, challenged,” “Baypamun diluted 1:36, challenged,” respectively).

At the maximum of the early disease (inflammatory) response on day 4 after inoculation, mice of group “naive, challenged” were very scruffy and clinically sick, while the “live C. psittaci B577 vaccinated, challenged” group appeared healthy, and BAYPAMUN®-treated groups were somewhat scruffy, but much less so that the “naive, challenged” group.

After day 4, all groups except group “naive, challenged” recovered quickly and appeared clinically healthy, while mice of group “naive, challenged” appeared progressively emaciated and developed pumping respiration. Three mice in this group were sacrificed on days 10 and 11 after inoculation (p.i.) prior to the scheduled date on day 12 p.i., since they would not have survived.

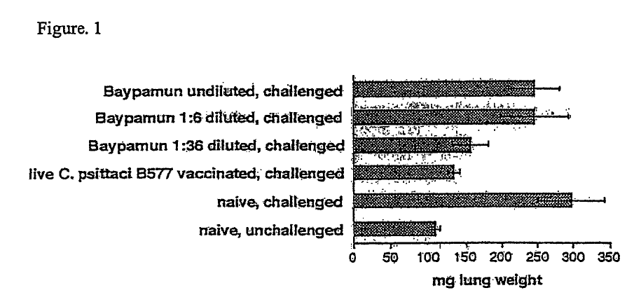

On day 12 p.i., all mice were sacrificed. The low lung weight and low lung weight increase (measure of disease intensity) of the group “live C. psittaci B577 vaccinated, challenged” reflects the typical protective immune response and a lung without macroscopic lesions and microscopic interstitial in-filtrate, but with prominent, microscopic peribronchiolar lymphocytic cuffs (FIG. 1). Mice of group “Baypamun 1:36 diluted, challenged” were essentially identical to group “live C. psittaci B577 vaccinated, challenged” with the exception of one mouse which had a lung with visible tissue consolidation and increased weight. Mice of the other BAYPAMUN® treated groups had bimodal responses, with some mice without any macroscopic lesions while others had clear areas of tissue consolidation due to interstitial pneumonia. However, these lesions generally were less severe than in the severely diseased “naive, challenged” group.

In conclusion, it was shown that Parapoxvirus has a dramatic protective effect against infections with strictly intracellular bacteria, especially with Chlamydia.

### REFERENCES

German patent application DE3504940
Cook, 1999. Antimicrobial therapy for Chlamydia pneumoniae: its potential role in atherosclerosis and asthma. J. Antimicrob. Chemother., 44(2):145-8.
Grayston et al., 1989. Current knowledge on Chlamydia pneumoniae, strain TWAR, an important cause of pneumonia and other acute respiratory diseases. Eur J Clin Microbiol Infect Dis., 8(3):191-202.
Haig and Fleming, 1999. Vet. Immunol. Immunopathol. 72: 81-86.
Haig et al., 1996. Vet. Immunol. Immunopathol. 54:261-267.
Inman et al., 2000. Chlamydia and associated arthritis. Curr Opin Rheumatol., 12(4):254-62.
Leinonen, 2000. Chlamydia pneumoniae and other risk factors for atherosclerosis. J Infect Dis., 181 Suppl 3:S414-416.
Moses and Sriram, 2001. An infectious basis for multiple sclerosis: perspectives on the role of Chlamydia pneumoniae and other agents. BioDrugs. 15(3):199-206.
Raupach and Kaufmann, 2001. Curr. Opin. Immunol. 13: 417-428.
Yirrel et al., 1994. British J. Dermatol. 130: 438-443.
Zecconi et al., 1999. Efficacy of a biological response modifier in preventing Staphylococcus aureus intramammary infections after calving. J. Dairy Sci., 82(10):2101-2107

1. Use of Parapoxvirus preparations for the manufacture of a pharmaceutical composition for the prophylaxis or for the treatment of conditions related to infection with strictly intracellular bacteria.

2. Use of claim 1, wherein the infection is with Chlamydia.

3. Use of claim 1, wherein the Parapoxvirus preparation comprises material from a Parapoxvirus ovis, or from a Parapoxvirus ovis strain NZ2, or from a Parapoxvirus ovis strain NZ7, or from a Parapoxvirus ovis strain NZ10, or from a Parapoxvirus ovis strain D1701, or from an orf virus, or an orf-11 virus.

4. Use of claim 1, wherein the condition related to infection with strictly intracellular bacteria is arteriosclerosis, and/or pneumonia, and/or multiple sclerosis, and/or asthma, and/or arthritis.

5. Use of claim 1, wherein the Parapoxvirus preparation comprises recombinant Parapoxvirus protein.

6. Method of treatment of conditions related to infections with strictly intracellular bacteria by administering to a subject in need a therapeutically effective dose of a Parapoxvirus preparation.

7. Method of claim 6, wherein the infection is with Chlamydia.

8. Method of claim 6, wherein the Parapoxvirus preparation comprises material from Parapoxvirus ovis, or from a Parapoxvirus ovis strain NZ2, or from a Parapoxvirus ovis strain NZ7, or from a Parapoxvirus ovis strain NZ10, or from a Parapoxvirus ovis strain D1701, or from an orf virus, or an orf-11 virus.

9. Method of claim 6, wherein the condition related to infection with strictly intracellular bacteria is arteriosclerosis, and/or pneumonia, and/or multiple sclerosis, and/or asthma, and/or arthritis.

10. Method of claim 6, wherein the Parapoxvirus preparation comprises recombinant Parapoxvirus protein.

11. Pharmaceutical composition for use in the treatment of prophylaxis of conditions related to infections with strictly intracellular bacteria, wherein said pharmaceutical composition comprises a Parapoxvirus preparation.

12. Pharmaceutical composition of claim 11, wherein the infection is with Chlamydia.

13. Pharmaceutical composition of claim 11, wherein the Parapoxvirus preparation comprises material from a Parapoxvirus ovis, or a Parapoxvirus ovis strain NZ2, or a Parapoxvirus ovis strain NZ7, or a Parapoxvirus ovis strain NZ10, or a Parapoxvirus ovis strain D1701, or an orf virus, or an orf-11 virus.

14. Pharmaceutical composition of claim 11, wherein the condition related to infection with strictly intracellular bacteria is arteriosclerosis, and/or pneumonia, and/or multiple sclerosis, and/or asthma, and/or arthritis.

15. Pharmaceutical composition of claim 11, wherein the Parapoxvirus preparation comprises recombinant Parapoxvirus protein.
